(19) United States
(12) Patent Application Publication
(10) Pub. No.: US 2009/0163933 A1
(43) Pub. Date: Jun. 25, 2009

(54) Cold Wax Ear Hair Removal Instrument

(75) Inventor: John DiMaio, Ossining, NY (US)

Correspondence Address: NORRIS, MCLAUGHLIN & MARCUS, 875 THIRD AVE, 18TH FLOOR, NEW YORK, NY, 10022, US

(73) Assignee: John DiMaio, Ossining, NY (US)

(21) Appl. No.: 11/961,002

(22) Filed: Dec. 20, 2007

(51) Int. Cl. A61B 17/50

(52) U.S. Cl. 606/134; 606/133

(57) ABSTRACT

An ear hair removal instrument including a head, at least a portion of which its outer surface is covered with cold wax. The head is shaped and sized to be receivable in the outer ear while preventing insertion into the middle ear. A handle is mounted at its distal end to the head. In use, the head of the instrument is inserted in the outer ear so that the cold wax disposed on the head comes into contact with the ear hair. While disposed in the outer ear, the ear hair in contact with the cold wax is extracted from its follicles by simultaneously rotating and pulling the instrument.

### BACKGROUND OF THE INVENTION

1. Field of the Invention

The present invention is directed to a hair removal instrument and, in particular, to an instrument for the removal of hair from the outer ear and entrance to the middle ear using cold wax.

2. Description of Related Art

As they grow older, men typically grow unwanted or undesirable hair in their ears. One conventional method for removal of unwanted ear hair includes the use of electric razors specially designed and configured for the shape of the ear. Shaving simply cuts the hair shaft proximate the skin rather than removing it from the follicle. The shaving of ear hair using an electric razor therefore has the disadvantage of growing back, often times thicker than before. This technique is free from any discomfort, however, it requires the user to maintain such grooming habits on a continuing basis at relatively short intervals, e.g., once a month, if not more often.

An alternative to conventional electric shaving implements include tweezing in which the hair is removed from the follicle. Tweezing is time consuming and causes some degree of discomfort that varies depending on each individual's tolerance for pain. Furthermore, the part of the body to which the hair is being removed is also a factor. It is difficult without contorting one's self in an awkward position for the individual to clearly see their own ear in order to tweeze unwanted hair themselves without the assistance of another. Some individuals live alone and thus cannot readily solicit another for assistance or perhaps are embarrassed to do so.

Electrolysis is similar to tweezing in that hair is removed from the follicle. However, unlike tweezing, electrolysis requires treatment by a professional who is certified or skilled in the technique and therefore requires the individual to go somewhere to have this performed. Not only is this embarrassing and time consuming, it is also relatively expensive.

Therefore, it is desirable to provide an improved instrument and method of use thereof for removal of ear hair at the follicle that is relatively inexpensive, disposable, relatively painless and easily performed in the privacy of one's own home without the need of assistance.

### SUMMARY OF THE INVENTION

One aspect of the present invention relates to an ear hair removal instrument including a head, at least a portion of which its outer surface is covered with cold wax. The head is shaped and sized to be receivable in the outer ear while preventing insertion into the middle ear. In addition, the instrument also includes a handle having a distal end and a proximal end with the head being mounted to the distal end of the handle.

Another aspect of the present invention is directed to an ear hair removal instrument including a spherical shaped head, at least a portion of which its outer surface is covered with cold wax. The head has a diameter in the range between approximately ⅜ inch and approximately ⅝ inch, preferably approximately ½ inch. A handle having a distal end and a proximal end is mounted at its distal end to the head. The instrument further including a base disposed coaxially with the handle between the distal and proximal ends and a cover securable to the base for enclosing the head to prevent contamination.

Yet another aspect of the present invention is directed to a method for using the ear hair removal instrument as described in either of the preceding paragraphs. First, the instrument is inserted in the outer ear so that the cold wax disposed on the head comes into contact with the ear hair. While disposed in the outer ear, the ear hair in contact with the cold wax is extracted from its follicles by simultaneously rotating and pulling the instrument.

### BRIEF DESCRIPTION OF THE DRAWINGS

The foregoing and other features of the present invention will be more readily apparent from the following detailed description and drawings of illustrative embodiments of the invention wherein like reference numbers refer to similar elements throughout the several views and in which:

FIG. 1 is a perspective view of an exemplary cold wax ear hair removal instrument in accordance with the present invention with its assembled cover;

FIG. 2 is an exploded perspective view of the ear hair removal instrument in FIG. 1 with the cover removed from its base;

FIG. 3 is a representative view showing use of the ear hair removal instrument by inserting the cold wax head into the outer ear so that it contacts the ear hair to be removed;

FIG. 4 is a representative view showing the simultaneous rotating and pulling of the ear hair removal instrument while disposed in the outer ear and in contact with the ear hair to be removed; and

FIG. 5 is a representative view showing the ear hair removal instrument being withdrawn from the ear with the removed ear hair remaining on the cold wax head.

### DETAILED DESCRIPTION OF THE INVENTION

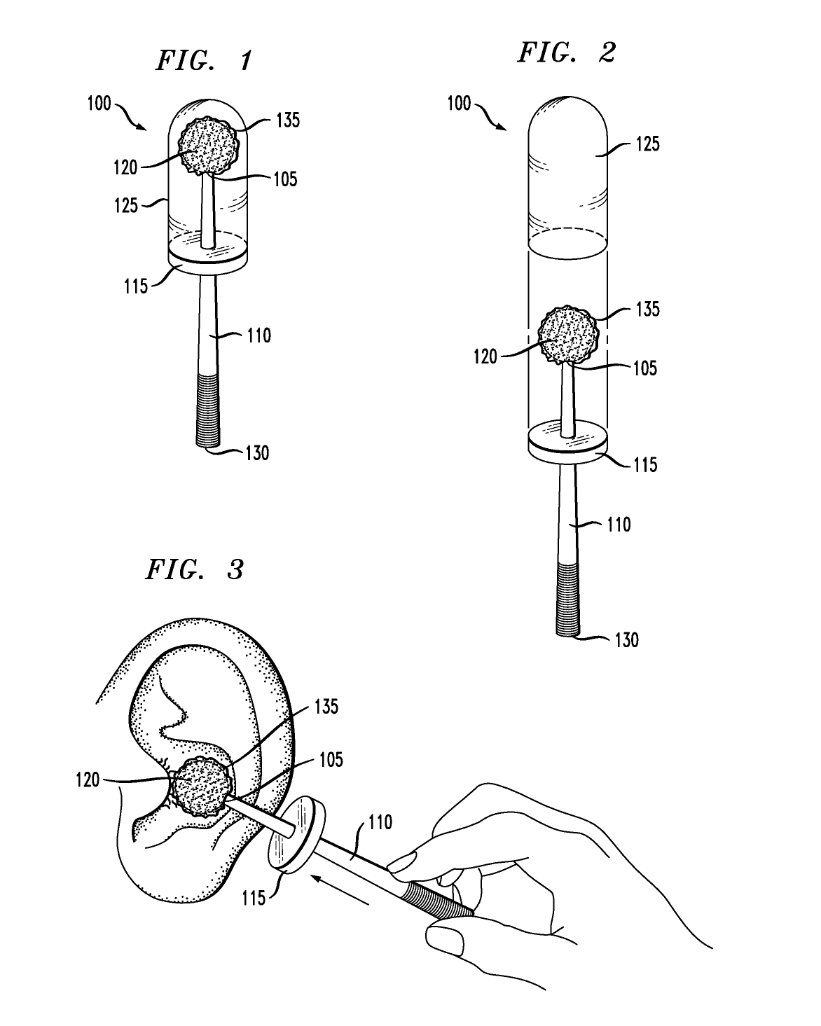

Referring to FIG. 1, the ear hair removal instrument 100 in accordance with the present invention includes a head 120 mounted, secured or attached to a shaft, stem, wand or handle 110. A ring, platform or base 115 is coaxially disposed between the head 120 and proximal end 130 of the instrument. Base 115 is designed to have an enlarged diameter relative to the handle 110 for receiving a cover, cap, enclosure or lid 125 thereby enclosing the head 120 to prevent contamination. A portion or the entire surface of the handle 110 may have a reduced slippage surface such as projections and/or recesses defined in the handle or a coating such as rubber or other non-slip surface. In the example shown in FIG. 1, a reduced slippage surface is provided on a portion of the handle 110 proximate its proximal end 130. Handle 110 is shown to have a tapered outer contour that has its largest diameter at its proximal end 130 and tapers towards its distal end 105. However, the handle may be cylindrically shaped or ergonomically designed to be contoured to the user's hand. Any shape handle 110 is contemplated and within the intended scope of the invention.

The material used to manufacture the head 120, handle 110 and base 115 may be the same or different. In a preferred embodiment the material used to manufacture these components is relatively inexpensive and thus the instrument may be disposed after a single application or use. Inexpensive materials that may be utilized are a polymer or cardboard. The instrument, if it is to be reused for multiple applications, can be manufactured from a more expensive material such as wood or metal. Whatever the material chosen for the handle 110 it is preferably rigid. On the other hand, the head 120 may be made from either a rigid, semi-rigid or a deformable material such as an elastically deformable material having an elastic shape memory (e.g., foam). The advantage of using an elastically deformable material having an elastic shape memory is that the head 120 when in use will conform to the shape of the ear and thus reach into more surfaces of the outer ear. Despite being deformable when in use, thereafter the head will automatically return to its original shape. Another advantage of using the deformable material is a greater degree of comfort when in use.

Head 120 is non-planar in shape, preferably spherical in shape, as shown in the Figures. However, alternative non-planar shapes may be used as desired such as ovoid, conical or other geometric shapes. Furthermore, the shape need not be symmetrical but instead may be asymmetrical. An outer coating or covering of cold wax 135 is applied to at least a portion of its outer surface, preferably to the entire surface of the head 120. The hair removal instrument is preferably manufactured and sold with the cold wax already applied to the outer surface of the head 120. The head is sized to be received within the outer ear but preferably not insertable due to its size and shape into the middle or inner ear where the user may cause damage. In the case of a spherical shaped head 120, its diameter is preferably between approximately ⅜ inch and approximately ⅝ inch, most preferably approximately ½ inch.

A cover, cap, enclosure or lid 125 encloses at least the head 120 to protect the cold wax from being contaminated or damaged by unintentionally coming into contact with a surface or debris in the air. Cover 125 is preferably made of a transparent material such as a polymer thereby allowing the user to readily view the head 120 therein. However, it is contemplated and within the intended scope of the present invention to use any material for the cover regardless of whether it is transparent or opaque so long as it prevents or substantially reduces the risk of contamination of the head 120. Cover 125, in the exemplary embodiment shown in FIGS. 1 and 2 is sized to be secured about the perimeter of base 115. FIGS. 1 and 2 depict cover 125 as terminating at the base 115, however, cover 125 may extend beyond the proximal tip 130 of the handle 110 thereby encasing the entire instrument. In an alternative embodiment, base 115 may be eliminated altogether and the hair removal instrument 100 encased in a blister pack or some other packaging design.

As previously mentioned, during manufacture, the head 120, base 114 and handle 110 are either manufactured as a single integral unit or assembled together if manufactured as separate components. Cold wax is applied to at least a portion, preferably the entire outer surface, of the head 120. The instrument with the cold wax applied to its head is then sold for use either individually or as multiple units.

In the case in which the instrument comprises multiple components (e.g., head 120, base 115, handle 110), they may be secured together using any conventional means. For example, the head 120 may be releasably secured to the handle 110 so that after use only the head, and not the handle 110, need be replaced. One mechanism for releasably securing the components is to have complementary threads on the distal end of the handle 110 and in the interior of an opening defined in the head 120 so that they may be screwed together. A releasable snap closure is another example. In still another example, the two components may be joined by tapering the distal end of the handle 110 so as to be received in an opening defined in the head and retained therein by a friction fit. Any securing mechanism, preferably a releasable securing mechanism can be employed.

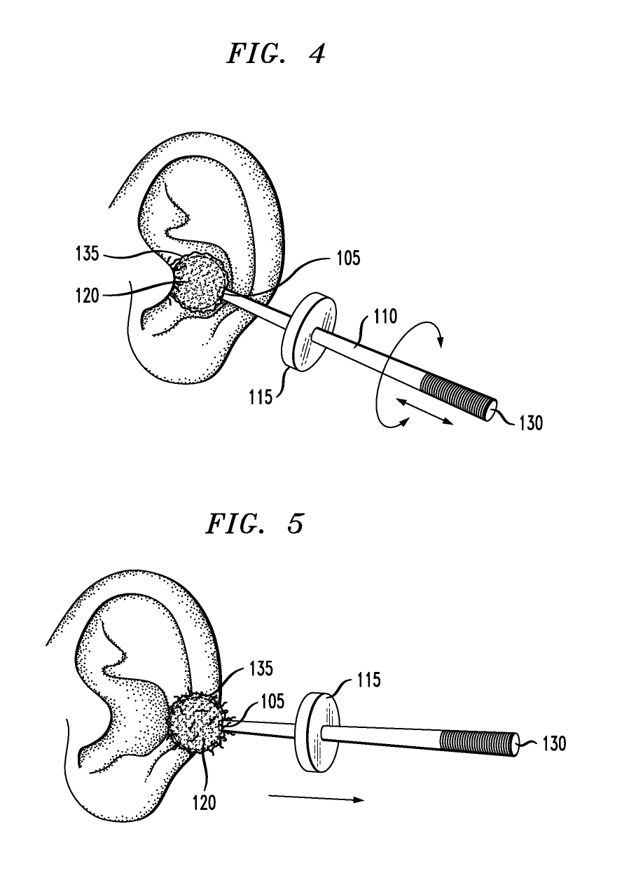

FIGS. 3-5 illustrate use of the ear hair removal instrument 100 in accordance with the present invention. The individual purchases the preassembled instrument with the cold wax coating applied to at least a portion of the outer surface of the head 120. The cover 125 is removed so as to gain access to the head 120. As shown in FIG. 3, while holding the instrument proximal the distal end of the handle 110, the user positions the head 120 so that it contacts the ear hair disposed in the outer ear. Upon contact, the ear hair sticks to the cold wax. While positioned in the outer ear, the user simultaneously rotates and pulls the instrument 100 causing the ear hair stuck to the cold wax to be extracted from its follicles. The instrument 100 with the ear hair stuck to the cold wax is removed from the outer ear. As previously mentioned, the instrument in its entirety or at least the head may be disposed of after use. In the alternative, the head 120 alone may be cleaned and cold wax reapplied to its outer surface in preparation for another treatment.

The Figures depict an embodiment in which the instrument 100 includes a single head 120 disposed at the distal end 115 of the handle 110. An alternative designed may comprise two heads, one disposed at each end of the handle 110, thus a single instrument can be utilized for removing the hair from both ears.

Thus, while there have been shown, described, and pointed out fundamental novel features of the invention as applied to a preferred embodiment thereof, it will be understood that various omissions, substitutions, and changes in the form and details of the devices illustrated, and in their operation, may be made by those skilled in the art without departing from the spirit and scope of the invention. For example, it is expressly intended that all combinations of those elements and/or steps that perform substantially the same function, in substantially the same way, to achieve the same results be within the scope of the invention. Substitutions of elements from one described embodiment to another are also fully intended and contemplated. It is also to be understood that the drawings are not necessarily drawn to scale, but that they are merely conceptual in nature. It is the intention, therefore, to be limited only as indicated by the scope of the claims appended hereto.

Every issued patent, pending patent application, publication, journal article, book or any other reference cited herein is each incorporated by reference in their entirety.

What is claimed is:

1. An ear hair removal instrument comprising:
a head, at least a portion of which its outer surface is covered with cold wax; the head is shaped and sized to be receivable in the outer ear while preventing insertion into the middle ear; and
a handle having a distal end and a proximal end, the head being mounted to the distal end of the handle.

2. The ear hair removal instrument in accordance with claim 1, wherein the head is non-planar in shape.

3. The ear hair removal instrument in accordance with claim 2, wherein the head is spherical in shape.

4. The ear hair removal instrument in accordance with claim 3, wherein the head has a diameter in the range from approximately ⅜ inch to approximately ⅝ inch.

5. The ear hair removal instrument in accordance with claim 4, wherein the diameter of the head is approximately ½ inch.

6. The ear hair removal instrument in accordance with claim 1, wherein the head is made of a polymer or cardboard.

7. The ear hair removal instrument in accordance with claim 1, wherein the head is made from a deformable material having an elastic memory shape.

8. The ear hair removal instrument in accordance with claim 1, wherein the hair removal substance covers the entire outer surface of the head.

9. The ear hair removal instrument in accordance with claim 1, further comprising another head mounted to the proximal end of the handle, at least a portion of which its outer surface is covered with the cold wax.

10. The ear hair removal instrument in accordance with claim 1, further comprising a cover for preventing contamination of the head.

11. The ear hair removal instrument in accordance with claim 10, further comprising a base coaxially disposed between the distal and proximal ends of the handle, the cover being securable to the base.

12. The ear hair removal instrument in accordance with claim 1, wherein the head and handle form an integral unit.

13. The ear hair removal instrument in accordance with claim 1, wherein the head is releasably securable to the distal end of the handle.

14. The ear hair removal instrument in accordance with claim 1, wherein at least a portion of the handle has a surface that reduces slippage.

15. An ear hair removal instrument comprising:
a spherical shaped head, at least a portion of which its outer surface is covered with cold wax; the head having a diameter in the range between approximately ⅜ inch and approximately ⅝ inch;
a handle having a distal end and a proximal end, the head being mounted to the distal end of the handle;
a base disposed coaxially with the handle between the distal and proximal ends; and
a cover securable to the base for enclosing the head to prevent contamination.

16. A method for removal of ear hair using an instrument that includes a head with at least a portion of which its outer surface is covered with cold wax, the head is shaped and sized to be receivable in the outer ear while preventing insertion into the middle ear, the instrument further including a handle mounted at its distal end to the head, the method comprising the steps of:
inserting the instrument in the outer ear so that the cold wax disposed on the head comes into contact with the ear hair; and
while disposed in the outer ear, extracting the ear hair in contact with the cold wax from its follicles by simultaneously rotating and pulling the instrument.

17. The method in accordance with claim 16, wherein the head is non-planar in shape.

18. The method in accordance with claim 17, wherein the head is spherical in shape.

19. The method in accordance with claim 18, wherein the head has a diameter in the range from approximately ⅜ inch to approximately ⅝ inch.

20. The method in accordance with claim 16, wherein the head is made from a deformable material having an elastic memory shape.
